(19) United States
(12) Patent Application Publication
(10) Pub. No.: US 2025/0120623 A1
(43) Pub. Date: Apr. 17, 2025

(54) BLOOD CHARACTERISTIC MEASURER AND MEASURING METHOD OF THE SAME AND MEASURING METHOD OF GLYCATED HEMOGLOBIN

(71) Applicant: AUO Corporation, HSIN-CHU (TW)

(72) Inventors: Chang-Po CHAO, HSIN-CHU (TW); Ya-Zhu YANG, HSIN-CHU (TW); Yen-Tang HUANG, HSIN-CHU (TW); Mei-Lien HUANG, HSIN-CHU (TW); Yan-Liang CHEN, HSIN-CHU (TW); Teng-Wei HUANG, HSIN-CHU (TW)

(21) Appl. No.: 18/530,258

(22) Filed: Dec. 6, 2023

(30) Foreign Application Priority Data
Oct. 17, 2023 (TW) 112139665

(51) Int. Cl. A61B 5/1455; A61B 5/00; A61B 5/145

(52) U.S. Cl. CPC A61B 5/14552; A61B 5/14546; A61B 5/7267; A61B 2560/0228; A61B 2562/0233

(57) ABSTRACT

A blood characteristic measurer includes multiple light sources, a light sensor and a processor. The multiple light sources emit multiple beams of light of different dominant lightening wavelength. The light sensor receives multiple reflected light beams due to the reflection of incident light from a skin surface. The processor is electrically coupled to the light sensor and produces the blood characteristic according to reconstructed spectra generated by the multiple reflected light beams and absorption coefficient spectra generated by the skin surface.

### CROSS-REFERENCE TO RELATED APPLICATION

This application claims priority to Taiwan Application Serial Number 112139665, filed Oct. 17, 2023, which is herein incorporated by reference in its entirety.

### BACKGROUND

### Field of Invention

The present disclosure relates to a blood characteristic measurer. More particularly, the present disclosure relates to a non-invasive blood characteristic measurer.

### Description of Related Art

Glycated hemoglobin, one of the blood characteristics, is formed by the combination of glucose concentrated in the blood and hemoglobin and is an indicator for tracking the patient's control of glucose. The blood characteristic measurer is quite common nowadays but some issues of the use of the current blood characteristic measurer still remain. For example, the current measuring method adopts an invasive way of obtaining a small drop of blood, which makes the patient uncomfortable and produces biological wastes. Moreover, the measuring time is so long that an instant result may not be obtained.

### SUMMARY

A blood characteristic measurer according to one embodiment of the present disclosure is provided and has the following elements: multiple light sources, a light sensor, and a processor. The light sources emit multiple incident light beams of different dominant lightening wavelengths. The light sensor senses multiple reflected light beams due to reflection of the incident light beams from a skin surface. The processor is electrically coupled to the light sensor and generates a blood characteristic value based on a reconstructed spectrum generated from the reflected light beams and a material absorption coefficient spectrum of the skin surface.

In some embodiments, the light sources includes a first light source, a second light source, and a third light source, wherein a dominant lightening wavelength of the first light source, a dominant lightening wavelength of the second light source, and a dominant lightening wavelength of the third light source are different from each other.

In some embodiments, the dominant lightening wavelength of the first light source, the dominant lightening wavelength of the second light source, and the dominant lightening wavelength of the third light source are a short-wavelength band of visible light, a long-wavelength band of visible light, and a near-infrared band respectively.

In some embodiments, the first light source, the second light source, and the third light source emit corresponding one of the incident light beams to the skin surface at different time points.

In some embodiments, the blood characteristic value is a glycated hemoglobin concentration value.

In some embodiments, the blood characteristic value is a blood oxygen concentration value.

In some embodiments, the processor is further calibrated, based on multiple physiological parameters, the blood characteristic value to generate a calibrated blood characteristic value.

A blood characteristic measuring method according to one embodiment of the present disclosure is provided and has following steps: receiving, by a processor, multiple signals associated with multiple reflected light beams to generate a reconstructed spectrum; generating, by a neural network model in the processor, a blood characteristic value based on the reconstructed spectrum; and calibrating, based on multiple physiological parameters, the blood characteristic value to generate a calibrated blood characteristic value.

In some embodiments, generating, by the neural network model in the processor, the blood characteristic value comprises: selecting multiple wavelength bands of the reconstructed spectrum to generate multiple material concentration values based on a material absorption coefficient spectrum of a skin surface that reflects the reflected light beams; and generating the blood characteristic value based on the material concentration values.

In some embodiments, an absorption coefficient of multiple materials, corresponding to the material concentration values, has a largest difference value within the wavelength bands in the material absorption coefficient spectrum.

In some embodiments, the physiological parameters include height data, weight data, or a combination thereof.

A glycated hemoglobin measuring method according to one embodiment of the present disclosure is provided and has following steps: training, based on multiple first training data, a first neural network model to output multiple reconstructed spectra, wherein the first training data include multiple real spectra; training, based on multiple second training data, a second neural network model to generate multiple first material concentration values, wherein the second training data include the reconstructed spectra and multiple second material concentration values corresponding to the reconstructed spectra; comparing the first material concentration values and the second material concentration values to adjust multiple setting parameters in the second neural network model; and adjusting, based on multiple physiological parameters, multiple parameters corresponding to multiple dense layers in the setting parameters in the second neural network model to generate a glycated hemoglobin value.

In some embodiments, training, based on the first training data, the first neural network model comprises: adjusting the first neural network model to generate a first set of setting parameters based on a first dataset corresponding to a first skin surface in the first training data; and adjusting the first neural network model to generate a second set of setting parameters based on a second dataset corresponding to a second skin surface, different from the first skin surface, in the first training data, wherein the first set of setting parameters is different from the second set of setting parameters.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1 is a schematic diagram of a blood characteristic measurer according to some embodiments in the present disclosure.

FIG. 2 is a flowchart of a blood characteristic measuring method for a blood characteristic measurer according to some embodiments in the present disclosure.

FIG. 3 is a schematic diagram of a neural network model for generating a reconstructed spectrum according to some embodiments in the present disclosure.

FIG. 4 is a reconstructed spectrum of a skin surface in a wavelength band of 400 nm to 1000 nm according to some embodiments in the present disclosure.

FIG. 5 is a schematic diagram of a neural network model for generating a blood concentration value and a water concentration value based on a reconstructed spectrum according to some embodiments in the present disclosure.

FIG. 6 is a material absorption coefficient spectrum of a skin surface in a wavelength band of 400 nm to 1000 nm according to some embodiments in the present disclosure.

FIG. 7 is a schematic diagram of a neural network model for generating a concentration value of hemoglobin carrying oxygen, a concentration value of hemoglobin without oxygen, and a glycated hemoglobin concentration value based on a reconstructed spectrum.

FIG. 8 is a flowchart of a glycated hemoglobin measuring method according to some embodiments in the present disclosure.

FIG. 9 is a schematic diagram of training a neural network model according to some embodiments in the present disclosure.

FIG. 10 is a schematic diagram of training a neural network model according to some embodiments in the present disclosure.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

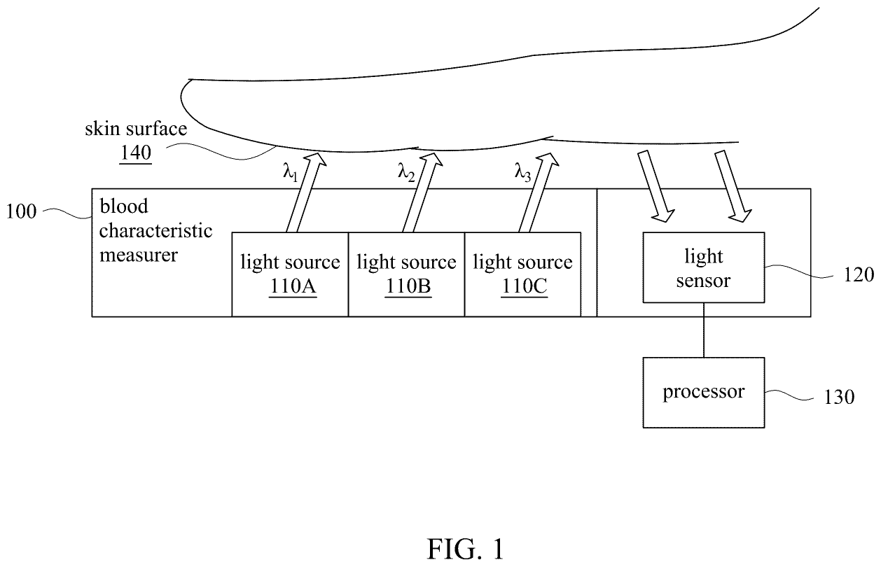

Reference is made to FIG. 1. FIG. 1 is a schematic diagram of a blood characteristic measurer 100 according to some embodiments in the present disclosure. The blood characteristic measurer 100 includes three light sources: a light source 110A, a light source 110B, and a light source 110C, a light sensor 120 and a processor 130.

The light sources 110A, 110B and 110C emit multiple incident light beams of different dominant lightening wavelengths to a skin surface 140. In some embodiments of FIG. 1, the skin surface 140 refers to a finger skin surface and in other embodiments the skin surface 140 refers to a wrist skin surface. The dominant lightening wavelengths of the light sources 110A, 110B and 110C are different from each other. For example, in some embodiments, the dominant lightening wavelength λ₁ of the light source 110A is in a short-wavelength band of visible light, the dominant lightening wavelength λ₂ of the light source 110B is in a long-wavelength band of visible light, and the dominant lightening wavelength λ₃ is in a near-infrared band (NIR). In some other embodiments, the dominant lightening wavelengths λ₁ to λ₃ of the light sources 110A to 110C may be 500 nm, 600 nm, and 800 nm respectively. In some embodiments, the light sources 110A, 110B and 110C emit corresponding one of the multiple incident light beams to the skin surface 140 at different time points. Alternatively stated, the light sources 110A, 110B and 110C emit multiple incident light beams at different time points separately.

In some embodiments, the light sources 110A, 110B, and 110C may be light emitting diodes (LED) and the number of the light sources is not limited therein.

The light sensor 120 senses multiple reflected light beams reflected from the skin surface 140 and converts a light signal(s) of the multiple reflected light beams into an electrical signal(s). The processor 130 is electrically connected to the light sensor 120 and receives the electrical signal associated with the reflected light from the light sensor 120. A neural network model in the processor 130 generates a blood characteristic value based on a reconstructed spectrum generated from the electrical signal and a material absorption coefficient spectrum of the skin surface 140. In some embodiments, the blood characteristic value includes a glycated hemoglobin concentration value and a blood oxygen concentration value. In some embodiments, the reconstructed spectrum may also be referred to as an absorption spectrum of the skin surface 140.

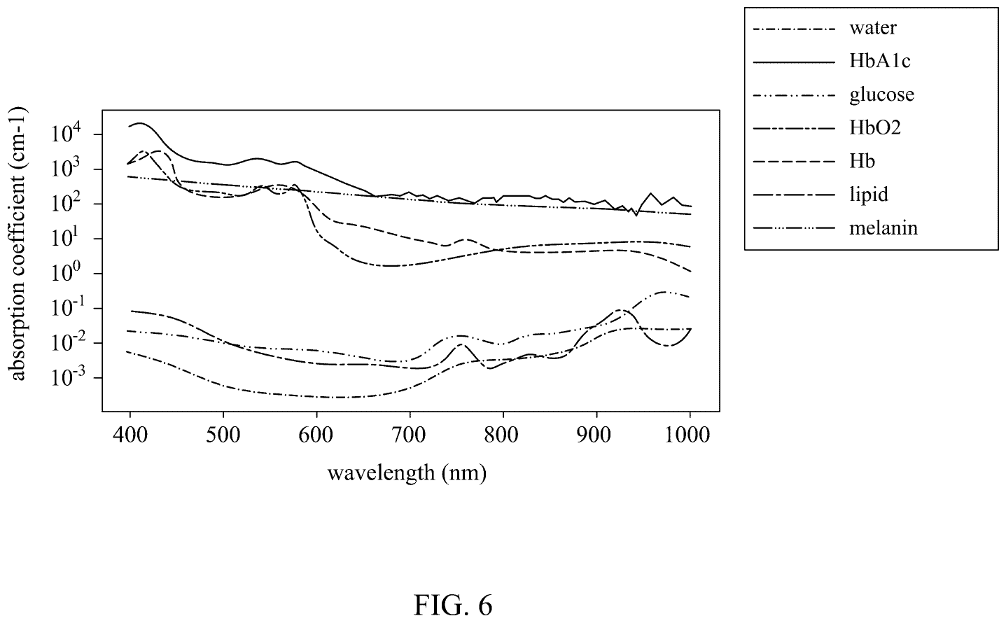

In some embodiments, the materials, of the material absorption coefficient spectrum of the skin surface 140, may be the materials presented in the absorption coefficient spectrum in FIG. 6, for instance, hemoglobin carrying oxygen or hemoglobin without oxygen.

In some embodiments, the light sensor 120 may be a photodiode (PD) and the number of the light sensor is not limited therein.

In some embodiments, the processor 130 includes a central processing unit (CPU), a micro control unit (MCU), a microprocessor, a digital signal processor (DSP), a programmable controller, an application specific integrated circuit (ASIC), a graphics processing unit (GPU), an arithmetic logic unit (ALU), a complex programmable logic device (CPLD), a field programmable gate array (FPGA), or another similar component or a combination of the foregoing components.

In some embodiments, the neural network model in the processor 130 includes a convolution neural network (CNN), a region with convolution neural network (R-CNN), a region proposal network (RPN), a recurrent neural network (RNN), a stacking-based deep neural network (S-DNN), a state-space dynamic neural network (S-SDNN), a deconvolution network, a deep belief network (DBN), a restricted Boltzmann machine (RBM), a fully convolutional network, a long short-term memory (LSTM), and classification network or another similar model or a combination of the foregoing models.

In some embodiments, the blood characteristic measurer 100 further includes an input interface, which enables the user to input multiple physiological parameters (e.g., height data, weight data, or a combination thereof) into the processor 130. In some embodiments, the neural network model in the processor 130 calibrates the blood characteristic value with the physiological parameters to generate a calibrated blood characteristic value. In some embodiments, the neural network model in the processor 130 may be further trained with the user's physiological parameters, so that the blood characteristic value (e.g., a glycated hemoglobin value) generated by the neural network model may further approach to the real physiological situation of the user. The related embodiments are about to be discussed in the following sections.

In some embodiments, the input interface may be a touch screen or any device suitable for receiving the user's input or instructions.

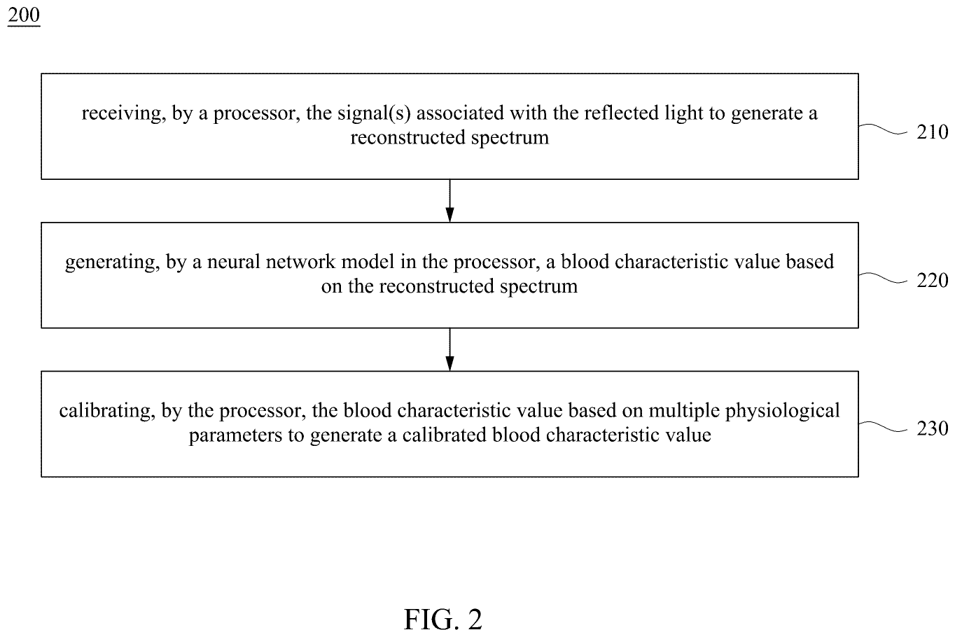

Reference is made to FIG. 2. FIG. 2 is a flowchart of a blood characteristic measuring method 200 for the blood characteristic measurer 100 according to some embodiments in the present disclosure. The description of the blood characteristic measuring method 200 is accompanied with the blood characteristic measurer 100 in FIG. 1. The blood characteristic measuring method 200 includes three steps 210, 220, and 230.

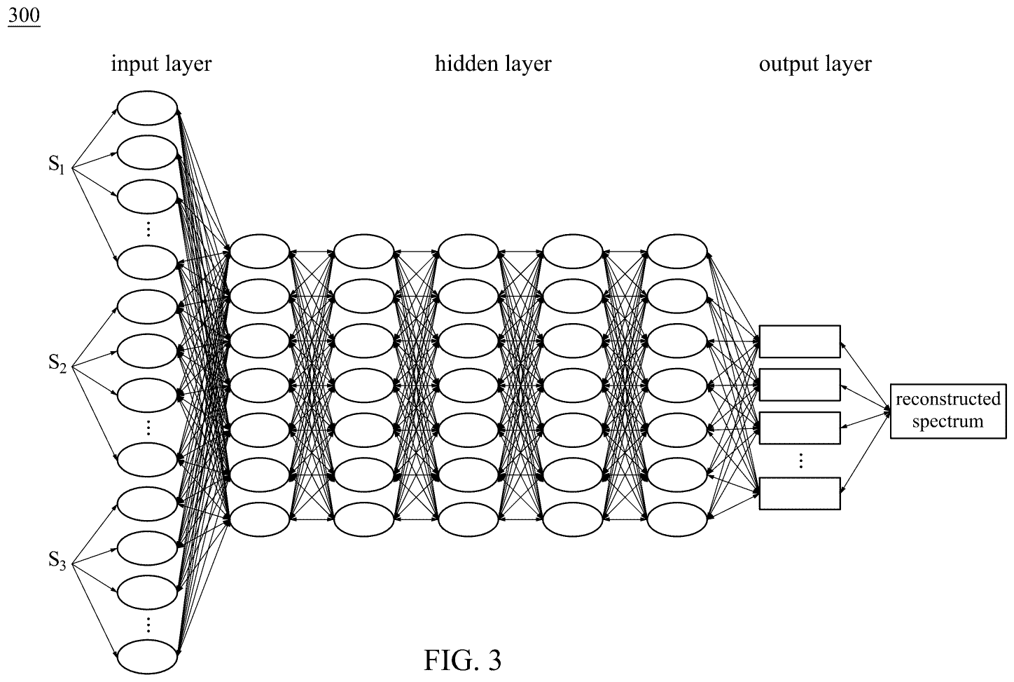

In step 210, as shown in FIG. 3, a neural network model 300 in the processor 130 receives signals S1, S2 and S3 that are associated with the reflected light to generate the reconstructed spectrum. The signals S1, S2 and S3 are electrical signals transmitted by the light sensor 120 to the processor 130. In some embodiments, for example, the signal S1 corresponds to the spectral power distribution, the spectral sensitivity, and the spectral reflectivity of the dominant lightening wavelength λ₁ or the like.

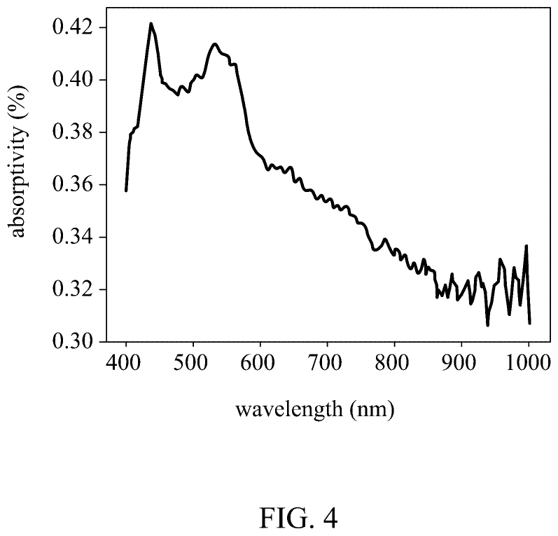

Specifically, the input layer of the neural network model 300 in FIG. 3 receives the signals S1, S2, and S3 as the inputs, mathematical operations of the hidden layer are performed to the signals S1, S2, and S3, and the output layer outputs a reconstructed spectrum as shown in FIG. 4. In some embodiments, the algorithmic process may be expressed in formula (1):

$${P}_{m} = {\int }_{Ω} s ( λ ) {l}_{m} ( λ ) {C}_{m} ( λ ) d λ \qquad ( 1 )$$

Pₘ is the reconstructed spectrum of the m-th light source, Q is the wavelength band of the light source, Iₘ(λ) is the wavelength band power distribution of the m-th light source, Cₘ(λ) is the spectral sensitivity of the m-th light source to the light sensor, s(λ) is the spectral reflectivity of the skin surface, and λ is the wavelength.

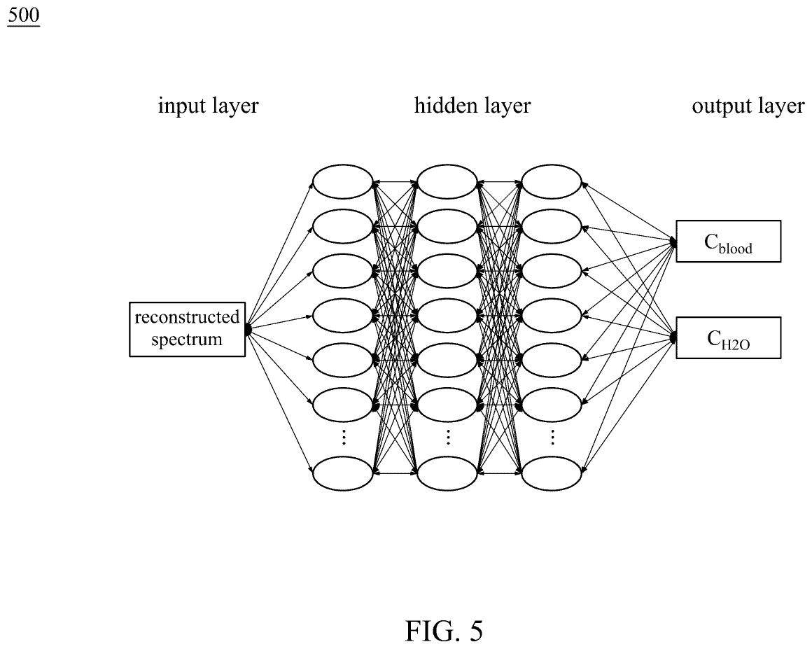

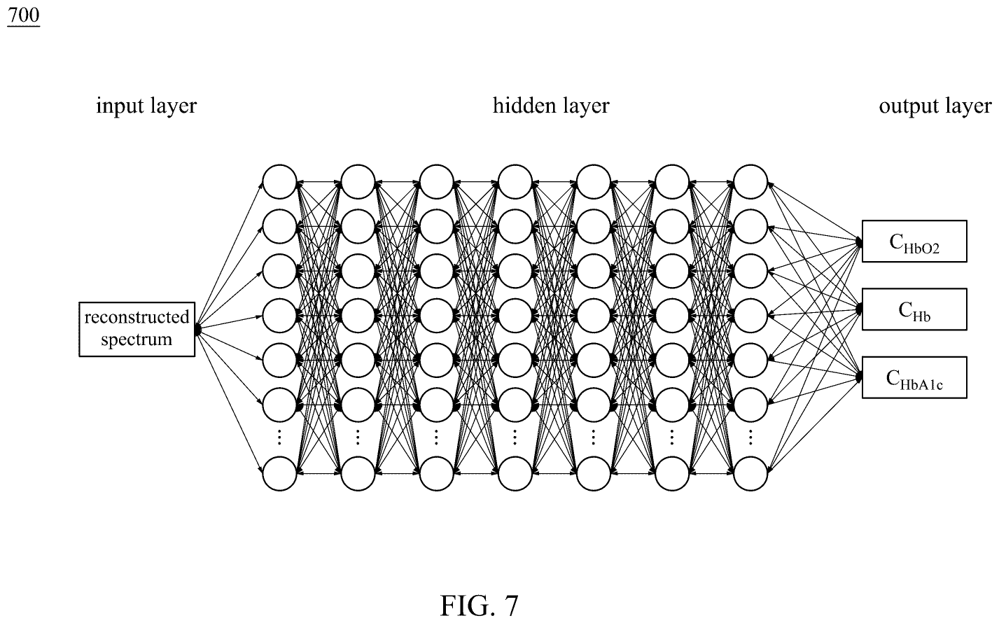

In step 220, another neural network model in the processor 130 (for instance, a neural network model 500 in FIG. 5 or a neural network model 700 in FIG. 7) generates the blood characteristic value based on the reconstructed spectrum in FIG. 4. In some embodiments, the neural network model 500 generating the blood characteristic value includes a step of selecting multiple wavelength bands of the reconstructed spectrum to generate material concentration values based on the material absorption coefficient spectrum of the skin surface 140 in FIG. 6, and a step of generating the blood characteristic value based on the material concentration values.

FIG. 4 is a reconstructed spectrum of the reflected light reflected from the skin surface 140 in the wavelength band of 400 nm to 1000 nm and FIG. 6 illustrates a material absorption coefficient spectrum of the skin surface 140 in the wavelength band of 400 nm to 1000 nm.

For example, based on the material absorption coefficient spectrum in FIG. 6, the neural network model 500 in the processor 130 determines whether the spectrum curve of a material has a discernible trend in a certain wavelength band compared to other materials. In some embodiments, the neural network model determines with the criterion that the absorption coefficient of the multiple materials has the largest difference value within the multiple wavelength bands in the material absorption coefficient spectrum. For example, in the wavelength band of 500 nm to 600 nm, the curve of hemoglobin carrying oxygen and the curve of water show a different trend, and, with respect to other wavelength bands, have the largest difference value in the wavelength band of 500 nm to 600 nm. In other embodiments, for example, in the wavelength band of 600 nm to 700 nm in FIG. 6, the curve of hemoglobin without oxygen and the curve of water show a different trend, and, with respect to other wavelength bands, have the largest difference value in the wavelength band of 600 nm to 700 nm.

The neural network model 500 selects the wavelength band of the reconstructed spectrum based on the stated method and generates multiple material concentration values based on the wavelength band of the reconstructed spectrum. In some embodiments, multiple material concentration values may be obtained from the reconstructed spectrum by using formula (2) and the formula (2):

$$A = {\sum }_{i = 1}^{N} {A}_{i} = {\sum }_{i = 1}^{N} {ε}_{i} \times {c}_{i} \times d = - log ( \frac{I}{{I}_{O}} ) \qquad ( 2 )$$

Aᵢ is the absorption coefficient of the material, N is the number of the type of the material, εᵢ is the absorption coefficient of the material, cᵢ is the concentration value of the material, d is the thickness, Iₒ is the intensity of the incident light, I is the intensity of the transmitted light.

In some embodiments, the detailed content of the formula (2) may be expressed in the formula (3) below:

$$A = \{ {C}_{bl\_dermis} * {C}_{HbO 2} * {μ}_{HbO 2} + {C}_{bl\_dermis} * {C}_{Hb} * {μ}_{Hb} + {C}_{bl\_dermis} * {C}_{HbA 1 c} * {μ}_{HbA 1 c} + ( 1 - {C}_{bl\_dermis} ) * {C}_{w\_dermis} * {μ}_{water} + ( 1 - {C}_{bl\_dermis} ) * ( 1 - {C}_{w\_dermis} ) * {μ}_{base} \} {*}_{dermis} + \{ {C}_{m} * β * {μ}_{pheumelanin} + {C}_{m} * ( 1 - β ) * {μ}_{eumelanin} + {C}_{w\_epidermis} * {μ}_{water} + ( 1 - {C}_{m} - {C}_{w\_epidermis} ) * {μ}_{base} \} * {d}_{epidermis} \qquad ( 3 )$$

where the description of the symbol in the formula (3) may be referred to the table 1.

TABLE 1

| Symbol | Content | Symbol | Content |
|---|---|---|---|
| C_{bl}_{_{—}}_{dermis} | blood volume | μ_{HbO2} | molar absorption |
| | fraction in | | coefficient of |
| | the dermis | | hemoglobin |
| | | | carrying oxygen |
| C_{HbO2} | volume fraction of | μ_{Hb} | molar absorption |
| | hemoglobin carrying | | coefficient of |
| | oxygen in the dermis | | hemoglobin |
| C_{Hb} | hemoglobin volume | μ_{bA1c} | molar absorption |
| | fraction in | | coefficient of |
| | the dermis | | glycated hemoglobin |
| C_{HbA1c} | glycated hemoglobin | μ_{water} | molar absorption |
| | volume fraction in | | coefficient of water |
| | the dermis | | |
| C_{w}_{_{—}}_{dermis} | water volume | μ_{base} | molar absorption |
| | fraction in | | coefficient of |
| | the dermis | | the baseline |
| Cₘ | melanin volume | μₚₕₑᵤₘₑₗₐₙᵢₙ | molar absorption |
| | fraction in | | coefficient of |
| | the dermis | | pheumelanin |
| C_{w}_{_{—}}_{epidermis} | water volume | μₑᵤₘₑₗₐₙᵢₙ | molar absorption |
| | fraction in the | | coefficient of |
| | epidermis | | eumelanin |
| d_{dermis} | thickness of | β | volume fraction of |
| | the dermis | | pheomelanin/ |
| | | | eumelanin |
| d_{epidermis} | thickness of the | A | total absorption |
| | epidermis | | coefficient |

According to the stated embodiments of selecting suitable wavelength bands to generate the concentration value of a specific material, the operation that generates the blood concentration value C_{blood} and the water concentration value C_{H2O} is about to be stated with reference to FIG. 5 and FIG. 6. FIG. 5 is a schematic diagram of the neural network model 500 which, based on the reconstructed spectrum, generates the blood concentration value C_{blood} and the water concentration value C_{H2O} according to some embodiments in the present disclosure. In some embodiments, the neural network model 500 selects the wavelength band of 900 nm to 1000 nm to analyze the reconstructed spectrum in order to generate the blood concentration value C_{blood} and the water concentration value C_{H2O}. Next, the generated blood concentration value C_{blood} and water concentration value C_{H2O} are calibrated based on the concentration range in the table 2 and then output from the output layer. For example, when the blood concentration value C_{blood} is outside the range of 0.2% to 7.0%, the neural network model 500 regenerates the blood concentration value C_{blood} and makes the blood concentration value C_{blood} within the range of 0.2% to 7.0%.

TABLE 2

| Symbol | Range |
|---|---|
| C_{blood} | 0.2-7.0% |
| C_{Hb} | 1.0-20.0% |
| C_{HbO2} | 80.0-99.9% |
| C_{HbA1c} | 3.0-15.0% |
| C_{H2O} | 70.0-83.0% |

Reference is made to FIG. 7. FIG. 7 is a schematic diagram of the neural network model 700 which based on the reconstructed spectrum, generates the concentration value of hemoglobin carrying oxygen, the concentration value of hemoglobin without oxygen, and the glycated hemoglobin concentration value, according to some embodiments in the present disclosure. In some embodiments, the neural network model 700 selects the wavelength band of 600 nm to 1000 nm to analyze the reconstructed spectrum in order to generate the concentration value of hemoglobin carrying oxygen C_{HbO2}, the concentration value of hemoglobin without oxygen C_{Hb}, and the glycated hemoglobin concentration value C_{HbA1c}. In the same way, the generated concentration value of hemoglobin carrying oxygen C_{HbO2}, concentration value of hemoglobin without oxygen C_{Hb}, and glycated hemoglobin concentration value C_{HbA1c} are calibrated based on the concentration range in the table 2 and are output from the output layer.

Multiple material concentration values are generated from the neural network model 500 in FIG. 5 and the neural network model 700 in FIG. 7 and then the blood characteristic value (e.g., a glycated hemoglobin concentration value and a blood oxygen concentration value) is further generated.

In step 230, the processor 130 calibrates the blood characteristic value to generate the calibrated blood characteristic value based on the physiological parameters. In some embodiments, the physiological parameters include height data, weight data, or a combination thereof. For instance, the blood characteristic values corresponding to the same reconstructed spectrum are adjusted to different calibrated blood characteristic values based on different height data, weight data, or a combination thereof. Alternatively stated, the blood characteristic measurer 100 may further provide a more accurate blood characteristic value based on the subject's different physiological parameters.

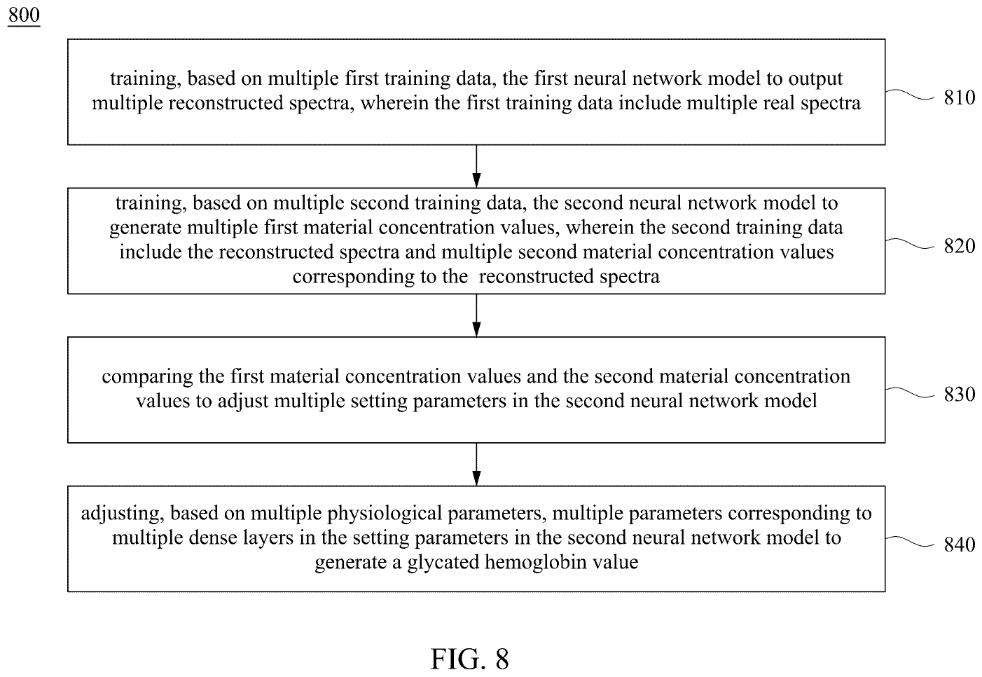

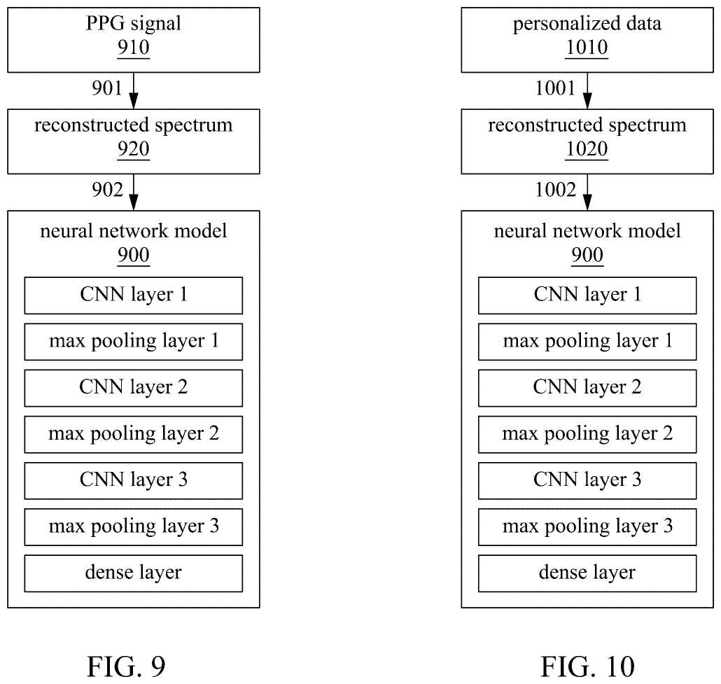

Reference is made to FIG. 8. FIG. 8 is a flowchart of a glycated hemoglobin measuring method 800 according to some embodiments in the present disclosure. The glycated hemoglobin measuring method 800 includes steps 810, 820, 830, and 840. In order to make the flow of the glycated hemoglobin measuring method 800 in FIG. 8 more understandable, please read the description of FIG. 8 also with reference to FIG. 9 and FIG. 10. FIG. 9 and FIG. 10 are schematic diagrams of training a neural network model in the glycated hemoglobin measuring method 800 according to some embodiments in the present disclosure. In some embodiments, the glycated hemoglobin measuring method 800 may realize the blood characteristic measurer 100 in FIG. 1. In some embodiments, the glycated hemoglobin measuring method 800 includes training the neural network model in the processor 130.

In step 810, the first neural network model is trained to output multiple reconstructed spectra based on multiple first training data and the multiple first training data include multiple real spectra. In some embodiments, the first neural network model may be the neural network model 300 outputting reconstructed spectra in FIG. 3. For example, the first training data include input data and the input data may be a PPG signal(s) that is generated by photoplethysmography (PPG) and stored in a large database (for instance, the multiple spectra generated from the reflection from the skin surface and measured by the blood characteristic measurer 100 in FIG. 1). The first training data also includes a hyperspectral image(s) (HSI), which is associated with the skin surfaces and generated by a hyperspectrometer, as a real spectrum. Then, as shown in FIG. 9, during the process 901, the parameters of the first neural network model are adjusted based on the difference between the input data and the real spectrum, so that when the training is completed, the reconstructed spectrum 920 that the first neural network model outputs based on the input data may meet the real spectrum from the hyperspectrometer.

In step 820, the second neural network model is trained to generate multiple first material concentration values based on multiple second training data. The multiple second training data include multiple reconstructed spectra and multiple second material concentration values corresponding to theses reconstructed spectra. In some embodiments, the second neural network model may correspond to the neural network model 900 in FIG. 9, the neural network model 500 or the neural network model 700. The first material concentration values generated by the second neural network model include the material concentration values in FIG. 5 and/or FIG. 7. In the process 902 in FIG. 9, the second neural network model is trained based on the second training data. In some embodiments, the second training data include the reconstructed spectrum generated by the first neural network model, the real glycated hemoglobin value that correspond to the reconstructed spectrum and is obtained from a blood glucose meter, and multiple material concentration values corresponding to the real glycated hemoglobin value. For example, the neural network model 900 receives the reconstructed spectrum and then generates the first material concentration values accordingly.

In step 830, the multiple first material concentration values and the multiple second material concentration values are compared to adjust multiple setting parameters in the second neural network model. In some embodiments, by comparing the multiple material concentration values corresponding to the real glycated hemoglobin value and the first material concentration values generated by the neural network model 900, the setting parameters in the neural network model 900 are adjusted to optimize the calculation result of the neural network model 900. Then, the glycated hemoglobin value is calculated based on the material concentration values output by the optimized neural network model 900. Finally, the calculated glycated hemoglobin value is calibrated with the real glycated hemoglobin value. In some embodiments, the aforementioned effect may be achieved by adjusting the setting parameters in each layer in the neural network model 900. In some embodiments, the multiple setting parameters include data weight, hyperparameters, training times or the like.

In some embodiments, the glycated hemoglobin measuring method 800 further includes using personalized data 1010 to train the first neural network model in the process 1001 as shown in FIG. 10. The operation of the processes 1001 and 1002 in FIG. 10 is similar to the operation of the processes 901 and 902 and therefore not repeated herein. In some embodiments, the personalized data include the PPG signal that corresponds to different physiological parameters.

In step 840, based on the multiple physiological parameters, multiple parameters corresponding to multiple dense layers in the setting parameters in the second neural network model are adjusted to generate a calibrated glycated hemoglobin value. In some embodiments, the neural network model 900 is trained with the physiological parameters, so that the glycated hemoglobin value generated by the neural network model 900 may correspond to different physiological parameters more correctly. For example, the dense layers in the neural network model 900 output the material concentration values. Therefore, by adjusting the parameters in the dense layers, the material concentration values output by the neural network model 900 are differentiated due to different physiological parameters and then the calibrated glycated hemoglobin value is calculated based on the material concentration values.

In some embodiments, the step 810 in the glycated hemoglobin measuring method 800 includes based on the data corresponding to a skin surface in the first training data, adjusting the first neural network model to generate a set of setting parameters. In some embodiments, the skin surface may refer to a finger skin surface and the data corresponding to the skin surface are multiple reconstructed spectra of the finger skin surface or other data and the setting parameters are data on the basis of the characteristic of the finger skin surface. In other embodiments, the skin surface may refer to a wrist skin surface and the data corresponding to the skin surface are multiple reconstructed spectra of the wrist skin surface or other data and the setting parameters are data on the basis of the characteristic of the wrist skin surface.

The blood characteristic measurer and the method in the present disclosure include the formation of the reconstructed spectrum on the basis of the incident light of different dominant lightening wavelengths, so that the spectral characteristics of multiple material concentration values may be precisely reflected and the neural network model may be effectively trained afterwards. Another feature of the present disclosure lies in that the individual user's physiological parameters may be put into the blood characteristic measurer to train the neural network model according to the user's physiological conditions, so that the measurement results may more agree with the user's physiological conditions.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A blood characteristic measurer, comprising:
a plurality of light sources configured to emit a plurality of incident light beams of different dominant lightening wavelengths;
a light sensor configured to sense a plurality of reflected light beams that are the plurality of incident light beams reflected from a skin surface; and
a processor electrically coupled to the light sensor and configured to generate a blood characteristic value based on a reconstructed spectrum, generated according to the plurality of reflected light beams, and a material absorption coefficient spectrum of the skin surface.

2. The blood characteristic measurer of claim 1, wherein the plurality of light sources include a first light source, a second light source, and a third light source, wherein a dominant lightening wavelength band of the first light source, a dominant lightening wavelength of the second light source, and a dominant lightening wavelength of the third light source are different from each other.

3. The blood characteristic measurer of claim 2, wherein the dominant lightening wavelength of the first light source, the dominant lightening wavelength of the second light source, and the dominant lightening wavelength of the third light source are a short-wavelength band of visible light, a long-wavelength band of visible light, and a near-infrared band respectively.

4. The blood characteristic measurer of claim 2, wherein the first light source, the second light source, and the third light source are configured to emit corresponding one of the plurality of incident light beams to the skin surface at different time points separately.

5. The blood characteristic measurer of claim 1, wherein the blood characteristic value is a glycated hemoglobin concentration value.

6. The blood characteristic measurer of claim 1, wherein the blood characteristic value is a blood oxygen concentration value.

7. The blood characteristic measurer of claim 1, wherein the processor is further configured to calibrate, based on a plurality of physiological parameters, the blood characteristic value to generate a calibrated blood characteristic value.

8. A blood characteristic measuring method, comprising:
receiving, by a processor, a plurality of signals associated with a plurality of reflected light beams to generate a reconstructed spectrum; and
generating, by a neural network model in the processor, a blood characteristic value based on the reconstructed spectrum, and
calibrating, based on a plurality of physiological parameters, the blood characteristic value to generate a calibrated blood characteristic value.

9. The blood characteristic measuring method of claim 8, wherein generating, by the neural network model in the processor, the blood characteristic value comprises:
selecting a plurality of wavelength bands of the reconstructed spectrum to generate a plurality of material concentration values based on a material absorption coefficient spectrum of a skin surface that reflects the plurality of reflected light beams; and
generating, based on the plurality of material concentration values, the blood characteristic value.

10. The blood characteristic measuring method of claim 9, wherein an absorption coefficient of a plurality of materials, corresponding to the material concentration values, has a largest difference value within the plurality of wavelength bands in the material absorption coefficient spectrum.

11. The blood characteristic measuring method of claim 8, wherein the plurality of physiological parameters include height data, weight data, or a combination thereof.

12. A glycated hemoglobin measuring method, comprising:
training, based on a plurality of first training data, a first neural network model to output a plurality of reconstructed spectra, wherein the plurality of first training data include a plurality of real spectra;
training, based on a plurality of second training data, a second neural network model to generate a plurality of first material concentration values, wherein the plurality of second training data include the plurality of reconstructed spectra and a plurality of second material concentration values corresponding to the plurality of reconstructed spectra;
comparing the first material concentration values and the second material concentration values to adjust a plurality of setting parameters in the second neural network model; and
adjusting, based on a plurality of physiological parameters, a plurality of parameters corresponding to a plurality of dense layers in the setting parameters in the second neural network model to generate a glycated hemoglobin value.

13. The glycated hemoglobin measuring method of claim 12, wherein training, based on the plurality of first training data, the first neural network model comprises:
adjusting the first neural network model to generate a first set of setting parameters based on a first dataset corresponding to a first skin surface in the plurality of first training data; and
adjusting the first neural network model to generate a second set of setting parameters based on a second dataset corresponding to a second skin surface, different from the first skin surface, in the plurality of first training data, wherein the first set of setting parameters is different from the second set of setting parameters.

14. The glycated hemoglobin measuring method of claim 12, wherein the plurality of physiological parameters include height data, weight data, or a combination thereof.

15. The glycated hemoglobin measuring method of claim 12, wherein a blood characteristic value is a glycated hemoglobin concentration value.
